(19) United States
(12) Patent Application Publication
(10) Pub. No.: US 2025/0064587 A1
(43) Pub. Date: Feb. 27, 2025

(54) TAVI Deployment Accuracy - Measures to Direct Balloon Inflation

(71) Applicant: St. Jude Medical, Cardiology Division, Inc., St. Paul, MN (US)

(72) Inventors: Peter J. Ness, Minneapolis, MN (US); Tyler Govek, St. Louis Park, MN (US); Neil Theisen, Champlin, MN (US); Hans Rieckmann, Minneapolis, MN (US)

(73) Assignee: St. Jude Medical, Cardiology Division, Inc., St. Paul, MN (US)

(21) Appl. No.: 18/802,263

(22) Filed: Aug. 13, 2024

Related U.S. Application Data
(60) Provisional application No. 63/578,810, filed on Aug. 25, 2023.

(51) Int. Cl. A61F 2/24; A61M 25/10

(52) U.S. Cl. CPC A61F 2/2433; A61M 25/1002; A61M 2025/1004

(57) ABSTRACT

A delivery device for delivering a prosthetic heart valve may include a balloon catheter shaft having a distal end, an inner shaft extending through the balloon catheter in a proximal-to-distal direction, an atraumatic distal tip positioned at a distal end of the inner shaft, and a balloon positioned between the distal end of the balloon catheter shaft and the atraumatic distal tip. In an uninflated condition of the balloon, the balloon may include a proximal bulge, a distal bulge, and an intermediate section having a diameter that is smaller than a diameter of the proximal bulge and smaller than a diameter of the distal bulge. The distal bulge may extend a first length in the proximal-to-distal direction, and the proximal bulge may extend a second length in the proximal-to-distal direction, the first length being different than the second length.

### CROSS-REFERENCE TO RELATED APPLICATIONS

This application claims priority to the filing date of U.S. Provisional Patent Application No. 63/578,810, filed Aug. 25, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement or implantation (“TAVR” or “TAVI”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to as a valve assembly) mounted to/within an expandable stent (the terms “stent” and “frame” are used interchangeably herein). In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts-including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When expanding a prosthetic heart valve into the native heart valve annulus, accurate deployment is typically an important indicator of the success of the prosthesis. For example, for an aortic heart valve replacement, the position of the prosthesis relative to the aortic annulus, as well as the extent to which the prosthesis extends into the left ventricular outflow tract (“LVOT”), can impact performance attributes of the prosthesis such as hemodynamics, PV leak, and the necessity of implanting a pacemaker with the prosthetic heart valve. Thus, it would be desirable to be able to increase the accuracy with which the prosthetic heart valve can be placed within the native valve annulus to optimize performance attributes of the prosthetic heart valve.

### SUMMARY OF THE DISCLOSURE

According to one aspect of the disclosure, a delivery device for delivering a prosthetic heart valve includes a balloon catheter shaft having a distal end, an inner shaft extending through the balloon catheter in a proximal-to-distal direction, an atraumatic distal tip positioned at a distal end of the inner shaft, and a balloon positioned between the distal end of the balloon catheter shaft and the atraumatic distal tip. The balloon may have an inflated condition and an uninflated condition. In the uninflated condition, the balloon may include a proximal bulge, a distal bulge, and an intermediate section between the proximal bulge and the distal bulge, the intermediate section having a diameter that is smaller than a diameter of the proximal bulge and smaller than a diameter of the distal bulge. The distal bulge may extend a first length in the proximal-to-distal direction, and the proximal bulge may extend a second length in the proximal-to-distal direction, the first length being different than the second length. The balloon catheter shaft may include an inflation lumen therein in fluid communication with an interior volume of the balloon such that pushing inflation media through the inflation lumen in the proximal-to-distal direction results in the inflation media entering the proximal bulge before entering the distal bulge. The first length may be between about 1, 1.1, 1.2, 1.3, 1.4, and about 1.5 times longer than the second length. In the inflated condition of the balloon, the diameters of the proximal bulge, distal bulge, and intermediate section may all be substantially equal.

According to another embodiment of the disclosure, a delivery device for delivering a prosthetic heart valve includes a balloon catheter shaft having a distal end, an inner shaft extending through the balloon catheter in a proximal-to-distal direction, an atraumatic distal tip positioned at a distal end of the inner shaft, and a balloon positioned between the distal end of the balloon catheter shaft and the atraumatic distal tip. The balloon may have an inflated condition and an uninflated condition. In the uninflated condition of the balloon, the balloon includes a proximal bulge, a distal bulge, and an intermediate section between the proximal bulge and the distal bulge, the intermediate section having a diameter that is smaller than a diameter of the proximal bulge and smaller than a diameter of the distal bulge. An inflation lumen modifier may be positioned on the inner shaft and may occupy a position within an interior volume of the balloon that is axially aligned with a position of the intermediate section. The inflation lumen modifier may be a solid cylindrical member, the inner shaft passing through an interior of the inflation lumen modifier. In the uninflated condition of the balloon, a total fillable interior volume of the intermediate section of the balloon may be smaller compared to the total fillable interior volume of the intermediate section of the balloon in an absence of the inflation lumen modifier. When the balloon transitions from the uninflated condition to the inflated condition, the proximal bulge, the intermediate section, and the distal bulge may expand at a substantially equal rate of expansion.

According to a further aspect of the disclosure, a delivery device for delivering a prosthetic heart valve includes a balloon catheter shaft having a distal end, an inner shaft extending through the balloon catheter in a proximal-to-distal direction, an atraumatic distal tip positioned at a distal end of the inner shaft, and a balloon positioned between the distal end of the balloon catheter shaft and the atraumatic distal tip. The balloon may have an inflated condition and an uninflated condition. In the uninflated condition of the balloon, the balloon may include a proximal bulge, a distal bulge, and an intermediate section between the proximal bulge and the distal bulge, the intermediate section having a plurality of folds or pleats so that a length of the intermediate section is greater than a length from a distal end of the proximal bulge to a proximal end of the distal bulge. The intermediate section may have a diameter that is smaller than a diameter of the proximal bulge and smaller than a diameter of the distal bulge. As the balloon transitions from the uninflated condition to the inflated condition, the plurality of folds or pleats of the intermediate section may unfold or unfurl. In the uninflated condition of the balloon, the balloon may have a first length between a proximal end of the proximal bulge and a distal end of the distal bulge, and in the inflated condition of the balloon, the balloon may have a second length between the proximal end of the proximal bulge and the distal end of the distal bulge, the first length being about equal to the second length. In the uninflated condition of the balloon, contact between the plurality of folds or pleats with an interior surface of a prosthetic heart valve may result in greater friction compared to friction between the interior surface of the prosthetic heart valve with an identically formed balloon that has an intermediate portion with a smooth outer surface without folds.

According to yet another aspect of the disclosure, a delivery device for delivering a prosthetic heart valve may include a balloon catheter shaft having a distal end, an inner shaft extending through the balloon catheter in a proximal-to-distal direction, an outer shaft extending over the balloon catheter shaft in the proximal-to-distal direction, an atraumatic distal tip positioned at a distal end of the inner shaft, and a balloon positioned between the distal end of the balloon catheter shaft and the atraumatic distal tip. The balloon may have an inflated condition and an uninflated condition. At least one spacer may be coupled to an outer surface of the inner shaft and may be in contact with an inner surface of the outer shaft so that the inner shaft is coaxial with the outer shaft. The at least one spacer may include a plurality of spacers positioned at spaced distances along the inner shaft. The at least one spacer may be fixed to the inner shaft. The spacer may be annularly shaped, with an interior circular hole through which the inner shaft passes, and an outer circumference in contact with the inner surface of the outer shaft. A distal end of the outer shaft may terminate proximal to a proximal end of the balloon.

According to still another aspect of the disclosure, a prosthetic heart valve system includes a delivery device and an expandable prosthetic heart valve. The delivery device may comprise a balloon catheter shaft having a distal end, an inner shaft extending through the balloon catheter in a proximal-to-distal direction, an atraumatic distal tip positioned at a distal end of the inner shaft, and a balloon positioned between the distal end of the balloon catheter shaft and the atraumatic distal tip. The balloon may have an inflated condition and an uninflated condition. The balloon may include a distal connection member extending radially outward from an outer surface of the balloon adjacent to a distal end of the balloon, and a proximal connection member extending radially outward from the outer surface of the balloon adjacent to a proximal end of the balloon. When the prosthetic heart valve receives the balloon therethrough, the distal connection member may contact an inflow portion of the prosthetic heart valve, and the proximal connection member may contact an outflow portion of the prosthetic heart valve. As the balloon transitions from the uninflated condition to the inflated condition, the distal connection member may remain in contact with the inflow portion of the prosthetic heart valve, and the proximal connection member may remain in contact with the outflow portion of the prosthetic heart valve.

### BRIEF DESCRIPTION OF DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIG. 3A illustrates a stent in a collapsed condition and positioned near a target deployment site.

FIG. 3B illustrates the stent of FIG. 3A after expanding.

FIG. 4A illustrates a stent in a collapsed condition and positioned near a target deployment site.

FIG. 4B illustrates the stent of FIG. 4A after expanding.

FIG. 5 is a cross-section of a distal end of a balloon catheter with pre-formed bulges in the balloon having different sizes when uninflated.

FIG. 6 is a cross-section of a distal end of a balloon catheter that includes an inflation lumen modifier.

FIG. 7A is a cross-section of a balloon for use with a balloon catheter according to another aspect of the disclosure.

FIG. 7B is a highly schematic view of the balloon of FIG. 7A before and after expansion.

FIG. 7C is a highly schematic view of an alternate version of the balloon of FIG. 7A before and after expansion.

FIG. 8 is a highly schematic cross-section of a delivery device deploying a prosthetic heart valve into a native aortic valve.

FIG. 9 is an isolated view of a balloon of a balloon catheter delivery device according to another aspect of the disclosure.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to the trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

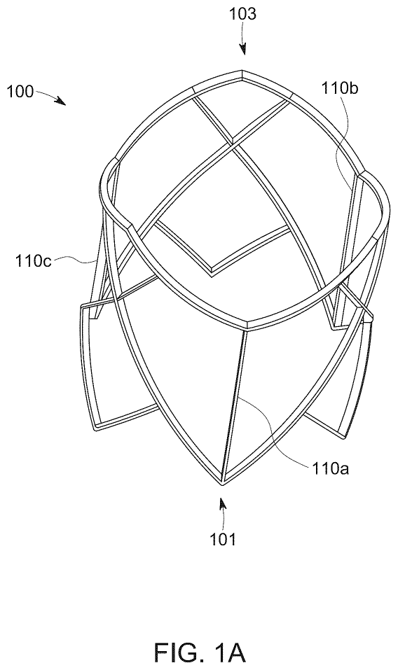

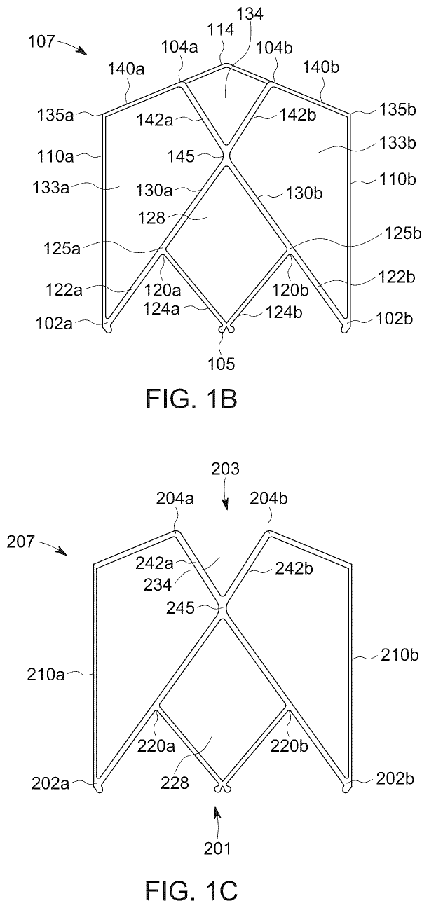

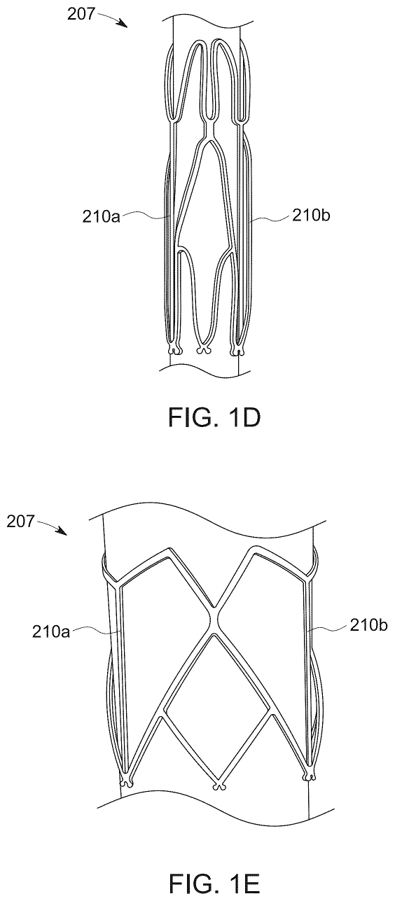

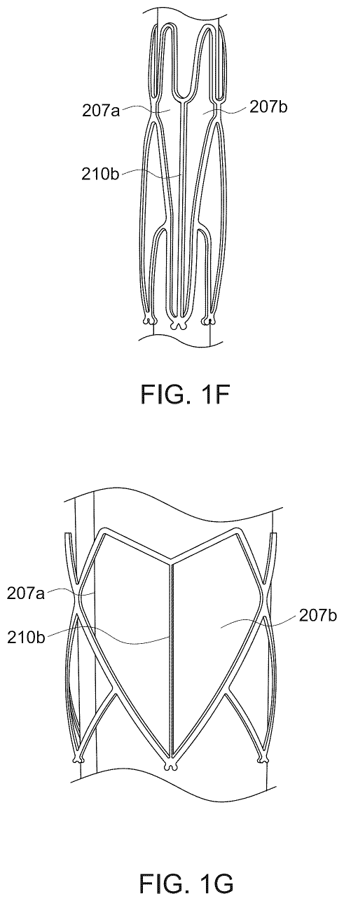

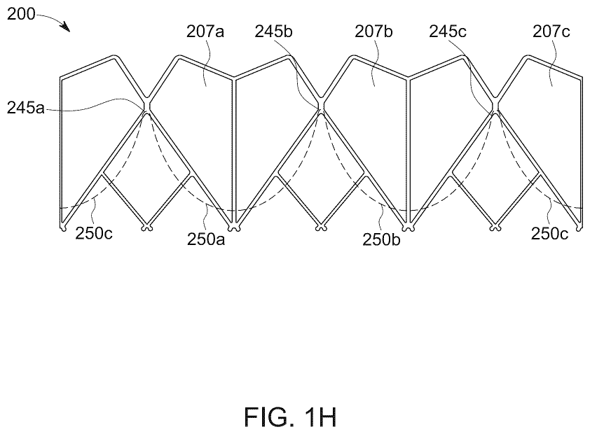

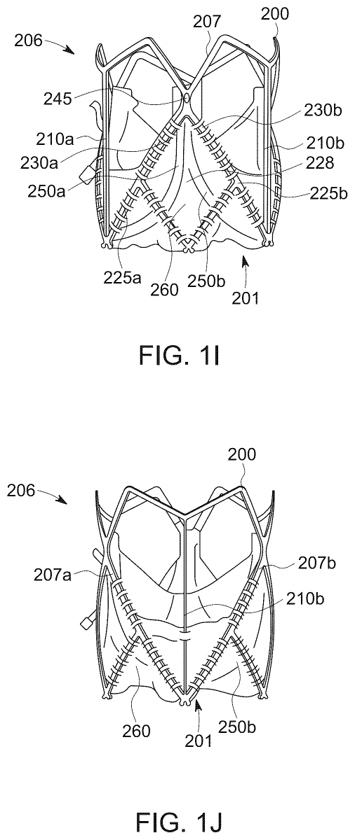

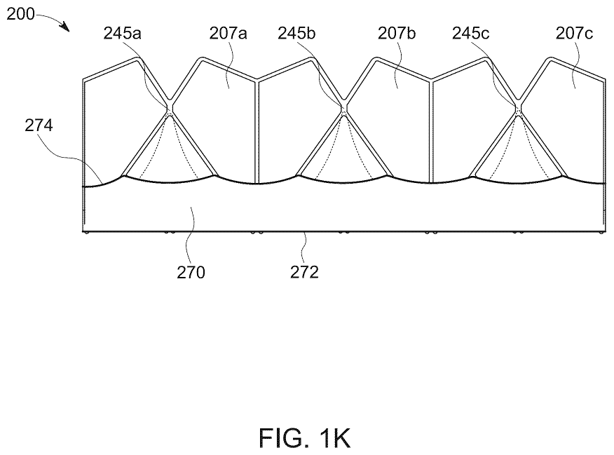

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135a and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as plastics, fabrics, and/or polymers, including ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help minimize or eliminate leakage around the outside of the valve (the latter known as paravalvular leakage or “PV” leakage). In the embodiment illustrated in FIGS. 1I-J, the cuff 260 only covers about half of the axial extent of stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. In some embodiments, the stent may be formed with cobalt chromium with additional metal or metal alloys such as nickel and/or molybdenum. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

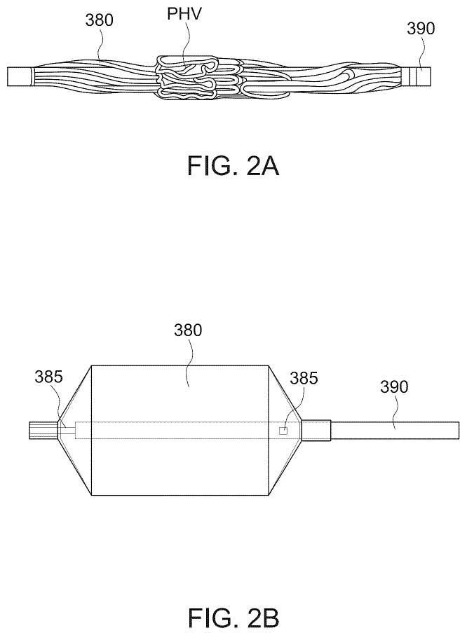

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

One potential complication with most expandable prosthetic heart valves is that typically, as the prosthetic heart valve radially expands into the native valve annulus during deployment, it also shortens axially. Another potential complication is the prosthetic heart valve axially shifting during insertion into the patient and/or while tracking (e.g. around the aortic arch) which could have adverse effects on the inflation accuracy. This is typically true of both self-expanding and balloon-expandable valves. The reason that this axial foreshortening may be problematic is that the axial position of certain valve elements relative to the native valve annulus prior to expansion will often not be the same as the axial position of those valve elements relative to the native valve annulus after expansion. In other words, if the prosthetic heart valve has a particular alignment that is shown under visualization (e.g. fluoroscopy) just prior to deployment, there will typically be at least some axial shifting of that relative alignment during deployment, and if such shifting is not minimized or otherwise compensated for, the resulting position of the deployed prosthetic heart valve may be non-optimal. In some circumstances, there may also be a rotational shift of the prosthetic heart valve relative to the native valve annulus during deployment if the balloon unwraps, unfolds, or untwists during expansion, which may make commissural alignment difficult.

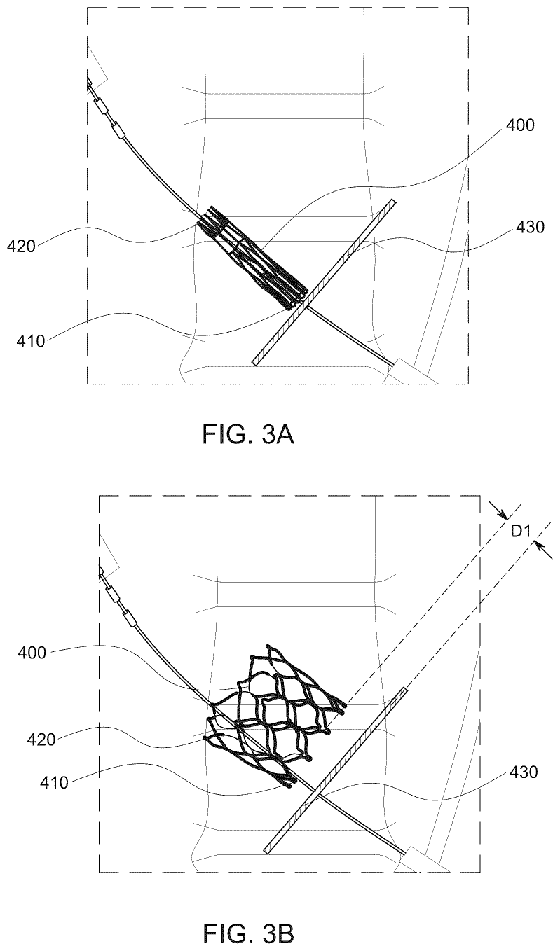

An example of the above-described axial shift is illustrated in FIGS. 3A-B. FIG. 3A illustrates a stent of a prosthetic heart valve collapsed over a balloon within a patient, with the figure being a fluoroscopic (e.g. x-ray) image so that only the metal stent 400 of the valve is easily visible. When deploying prosthetic heart valves, it is typically important that the inflow end of the prosthetic heart valve has a desired alignment with respective to the native valve annulus into which it is deployed. In FIGS. 3A-B, the desired target position of the inflow end 410 of the prosthetic heart valve is indicated by target line 430. The positioning of the outflow end 420 relative to the native valve annulus is typically less important (although not unimportant) compared to the alignment of the inflow end 410. In FIG. 3A, the stent 400 is still collapsed over a balloon and the inflow end 410 is exactly aligned with the target line 430. However, as deployment occurs, the stent 400 radially expands and axially foreshortens, so that after deployment, the inflow end 410 of the stent 400 is now a spaced distance DI away from the target line 430. In other words, because axially foreshortening occurred, and because that axial foreshortening was not (1) minimized and/or (2) compensated for otherwise, the initial relative positioning of the stent 400 with the anatomy (while collapsed) was not a reliable indicator of the final positioning of the stent 400 relative to the anatomy (when expanded). One way to compensate for this change in positioning is to try to advance the inflow end 410 of the stent 400 beyond the target line 430 prior to deployment, with the hope that compensated distance will equal the axial shift of the inflow end 410 so that the stent 400 ends up in the desired position after deployment. However, this may not be a particularly reliable method and may lead to inconsistent results. It would be preferable to have features in the prosthetic heart valve itself and/or on the delivery device that result in more accurate placement of the prosthetic heart valve during deployment and minimize the need for subjective positioning compensation at the beginning of deployment.

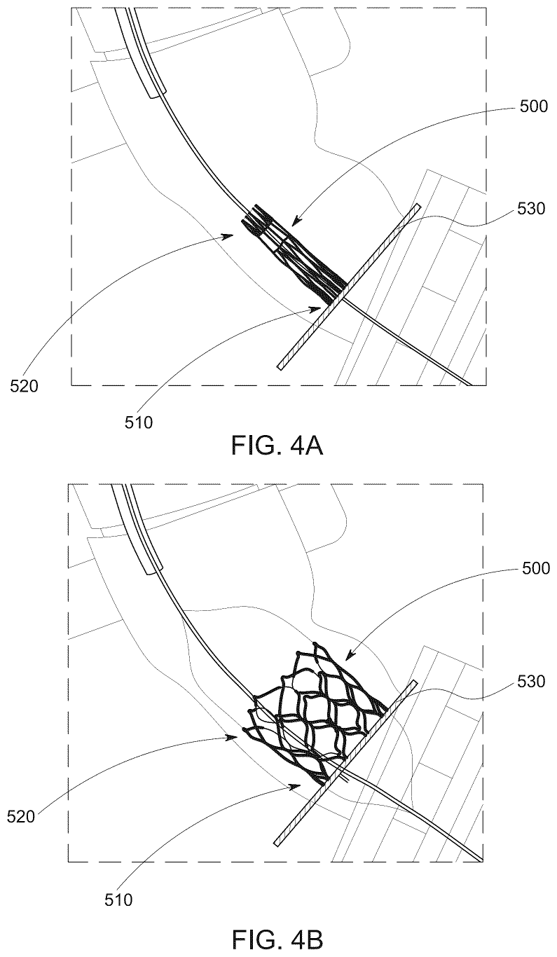

For example, FIGS. 4A-B illustrate one example of a desired deployment of a stent 500 of a prosthetic heart valve that is deployed via balloon expansion. FIG. 4A illustrates stent 500 of a prosthetic heart valve collapsed over a balloon within a patient, with the figure being a fluoroscopic (e.g. x-ray) image so that only the metal stent 500 of the valve is easily visible. Similar to FIGS. 3A-B, in FIGS. 4A-B the desired target position of the inflow edge 510 of the prosthetic heart valve is indicated by target line 530. In FIG. 4A, the stent 500 is still collapsed over a balloon and the inflow edge 510 is exactly aligned with the target line 530. As deployment occurs (and the balloon inflates), the stent 500 radially expands and axially foreshortens. However, unlike the situation of FIGS. 3A-B, the stent 500 of FIGS. 4A-B expands so that, after deployment, the inflow edge 510 of the stent 500 is still exactly (or nearly exactly) aligned with target line 530. One way in which this result is achievable is because, as the balloon expands, the outflow section 520 of the stent will foreshorten significantly more easily than the inflow section 510. In other words, the inflow edge 510 of the stent 500 may be used, prior to expansion or deployment, as a reliable visual indicator of where the inflow edge of the inflow section 510 of the stent 500 will be positioned after expansion or deployment. This reduces or eliminates the need to otherwise compensate for the axial foreshortening, for example by advancing the stent 500 “beyond” the target line 530 pre-deployment to try to achieve correct final positioning of the inflow end of the stent 500 at the target line 530 after deployment. This may eliminate significant guesswork from the deployment procedure, and generally increase the accuracy of the final axial positioning of the stent relative to the native valve annulus after deployment.

Although the designs of a prosthetic heart valve (and particularly the frame thereof) may be modified in ways to try to achieve the deployment configuration of FIGS. 4A-B, the delivery device itself may be designed to assist with achieving the deployment configuration of FIGS. 4A-B, or to achieve other desired stability of the prosthetic heart valve during deployment. Embodiments described below may assist in achieving at least some of these goals.

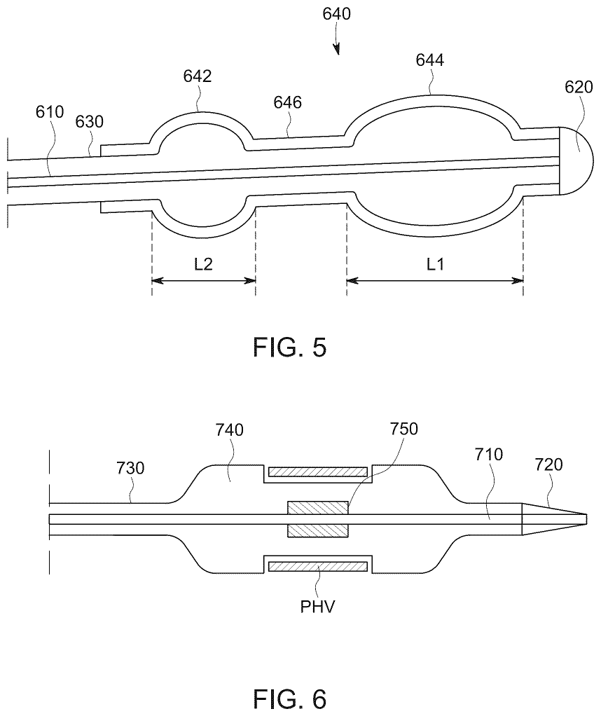

FIG. 5 illustrates a cross-section of a distal end of a delivery device for a balloon-expandable prosthetic heart valve. The delivery device may include an inner lumen or shaft 610 (for example sized to receive a guidewire therethrough) that is coupled, at its distal end, to an atraumatic distal tip or nosecone 620. The delivery device may also include a shaft 630 that has a distal end coupled to a proximal end of a balloon 640, with the distal end of the balloon 640 coupled to nosecone 620. In order to fill the balloon 640 to deploy the prosthetic heart valve mounted thereon, fluid may be passed into the interior of the balloon 640, for example by passing the fluid in the distal direction (towards the right in the view of FIG. 5) through a lumen of the shaft 630.

The balloon 640 of FIG. 5 is shown in a mostly or entirely uninflated condition. For example, the balloon 640 may be heat treated, or otherwise formed, so that the balloon 640 includes “pre-pillowed” sections that have bulging shapes even prior to fluid being passed into the balloon 640. In the illustrated embodiment, the balloon 640 includes a proximal pillow or bulge 642, a distal pillow or bulge 644, and an intermediate section 646 that connects the proximal bulge 642 and distal bulge 644. In the deflated or uninflated condition of the balloon 640, the proximal bulge 642 and distal bulge 644 may extend radially outwardly from the central longitudinal axis of the guidewire shaft 610 a greater distance than the intermediate section 646. With this configuration, the prosthetic heart valve may be mounted mostly or completely over the intermediate section 646 for delivery, and the proximal bulge 642 and distal bulge 644 may function as shoulders to help ensure the prosthetic heart valve remains securely in place during delivery. In other words, the outflow end of the prosthetic heart valve may contact the distal end of the proximal bulge 642, while the inflow end of the prosthetic heart valve may contact the proximal end of the distal bulge 644 (similar to the configuration shown in FIG. 6) to help the prosthetic heart valve retain its initial axial positioning relative to the balloon 640 during delivery. The distal bulge 644 may also provide edge protection to the leading edge (the inflow end for transfemoral delivery) of the prosthetic heart valve, for example to help avoid the inflow edge of the prosthetic heart valve from contacting native tissue (such as the interior wall of the aortic arch) which might otherwise cause the prosthetic heart valve to be displaced relative to the balloon 640.

If two equal-sized bulges 642, 644 are provided, and inflation media enters the balloon 640 from the proximal end of the balloon 640 (e.g. via the distal end of the shaft 630), the bulges 642, 644 may not expand at the same rate. This is particularly true for prosthetic heart valves that have relatively stiff inflow sections (which may be particularly suited to anchoring in the native valve annulus) and relatively flexible outflow sections (which may result from minimizing structure at the outflow end to provide maximum coronary artery clearance). Prosthetic heart valves that have inflow sections that are stiffer than their outflow sections are described in more detail in U.S. Provisional Patent Application No. ______, titled “TAVI Deployment Accuracy—Stent Frame Improvements” and filed on, ______, 2023, the disclosure of which is hereby incorporated by reference herein. In other words, if the inflow section of the prosthetic heart valve is more resistant to expansion, two equal-sized bulges 642, 644 may result in the outflow portion of the prosthetic heart valve to expand more quickly than the inflow section. Further, because the outflow portion of the prosthetic heart valve is positioned proximal to the inflow portion, the problem may be exacerbated because the fluid will enter the proximal bulge 642 prior to entering the distal bulge 644. If the bulges 642, 644 of the balloon 640 expand at different rates during deployment of the prosthetic heart valve, the prosthetic heart valve may expand in an uncontrolled and/or unintended way which may result in inaccurate positioning of the prosthetic heart valve within the native valve annulus. In some embodiments, it may be preferable for the bulges 642, 644 to expand at substantially equal rates as inflation media passes into the balloon 640 from the proximal end of the balloon 640. In order to achieve such equal rate of expansion, the distal bulge 644 may be provided with a length L1 that is greater than the length L2 of the proximal bulge 642. In the illustrated embodiment, the increased length L1 versus length L2 also results in the interior volume of distal bulge 644 being greater than the interior volume of proximal bulge 642 when the balloon 640 is in the uninflated condition. For example, in some embodiments, the distal bulge 644 may have a length L1 that is between 10% and 200% greater than the length L2, including about 25%, about 50%, about 75%, and about 100% greater. Similarly, in the uninflated conditions, the distal bulge 644 may have an internal volume that is between 10% and 200% greater than the internal volume of the proximal bulge 642, including about 25%, about 50%, about 75%, and about 100% greater. In one example, the length L1 is about 50% greater than the length L2, and as inflation media is pushed into the balloon 640 from the proximal end of the balloon 640, the proximal bulge 642 and the distal bulge 644 will expand at about the same rate. It should be understood that, during expansion of the balloon 640, the intermediate portion 646 will also expand. With substantially even rates of expansion of different portions of the balloon 640, the prosthetic heart valve mounted on the balloon 640 may be deployed with greater predictability.

In other embodiments, the sizes of the proximal bulge 642 and distal bulge 644 may be manipulated to provide preferential filling of either bulge before the other, which may be utilized to create specific deployment configurations of the prosthetic heart valve. For example, the “crimp zone” of the balloon 640, which may be the intermediate portion 646 between the two bulges 642, 644, may be further opened or further occluded to control the amount of flow resistance involved in inflating the distal bulge 644. Greater occlusion will increase the resistance of flow to the distal bulge 644, whereas less occlusion will decrease the resistance of flow to the distal bulge 644. In some embodiments, the design of the cone angles of the balloon 640 can impact the preferential filling of the proximal vs. distal balloon portions. As used herein, the term “cone angle” generally refers to the angle of the sloped region between the balloon leg and the main body of the balloon. Steeper cone angles generally have a thicker wall due to the nature of blow molding the balloon which may result in more resistance to expansion compared to portions of the balloon 640 with thinner walls. It should be understood that, after balloon 640 has been filled with inflation media, the outer diameters of the proximal bulge 642, distal bulge 644, and intermediate section 648 may all be substantially equal.

FIG. 6 illustrates a cross-section of a distal end of a delivery device for a balloon-expandable prosthetic heart valve according to another aspect of the disclosure. The delivery device may include an inner lumen or shaft 710 (for example sized to receive a guidewire therethrough) that is coupled, at its distal end, to an atraumatic distal tip or nosecone 720. The delivery device may also include a shaft 730 that has a distal end coupled to a proximal end of a balloon 740, with the distal end of the balloon 740 coupled to nosecone 720. In order to fill the balloon 740 to deploy the prosthetic heart valve mounted thereon, fluid may be passed into the interior of the balloon 740, for example by passing the fluid in the distal direction (towards the right in the view of FIG. 6) through a lumen of the shaft 730.

Similar to the balloon 640 of FIG. 5, the balloon 740 of FIG. 6 may, in an uninflated condition, form two bulges or “pillows” on each side of an intermediate portion of the balloon. However, in the particular embodiment of FIG. 6, each bulge has a substantially equal size. FIG. 6 also shows the prosthetic heart valve PHV crimped over the intermediate portion of the balloon 740, more clearly illustrating how the bulges pre-formed into the balloon 740 may help with maintaining the prosthetic heart valve PHV in the desired position relative to the balloon 740 during delivery, while also providing active protection of the leading edge of the prosthetic heart valve PHV during delivery. For example, FIG. 6 shows how the terminal inflow and outflow edges of the prosthetic heart valve PHV are in direct contact with complementary shoulders of each pre-formed bulge of the balloon 740 when the balloon 740 is in the uninflated condition.

As with the embodiment of FIG. 5, in order to fill the balloon 740 of FIG. 6 for deployment of the prosthetic heart valve PHV, the user may pass inflation media distally into the balloon 740, for example via an inflation lumen between the guidewire shaft 710 and balloon catheter shaft 730. In the illustrated embodiment, an inflation lumen modifier 750 is provided within the balloon 740. In this particular example, the inflation lumen modifier 750 is positioned within the intermediate portion of the balloon 740 (e.g., between the two pre-formed bulges) and around the guidewire shaft 710, for example by being fixed to the guidewire shaft 710. In the illustrated embodiment, the inflation lumen modifier 750 is a solid cylindrical member with the guidewire shaft 710 passing through an interior of the inflation lumen modifier 750. The inflation lumen modifier 750 may be formed of any suitable material, including for example a soft polymer that can be overmolded onto the balloon inner catheter shaft or guidewire shaft 710, including for example 25D-45D Pebax, and effectively acts as a strategically placed void filler. In other words, as shown in FIG. 6, the inclusion of the inflation lumen modifier 750 reduces the amount of open volume around the intermediate portion of the balloon 740. This additional, solid material will increase resistance to fluid flowing into the distal end of the balloon 740. In the absence of the inflation lumen modifier 750, resistance to flow will be decreased, and the distal end of the balloon 740 would fill more quickly than in the presence of the inflation lumen modifier. Thus, the size and position of the inflation lumen modifier 750 may be chosen to achieve the desired resistance to fluid flow, and thus the desired rate at which the distal end of the balloon 740 fills and expands relative to the proximal end of the balloon 740. With the particular embodiment illustrated in FIG. 6, the position of the inflation lumen modifier 750 may help ensure that the balloon 740 expands relatively uniformly as inflation media passes into the balloon 740, which may in turn result in more predictable expansion of the prosthetic heart valve PHV.

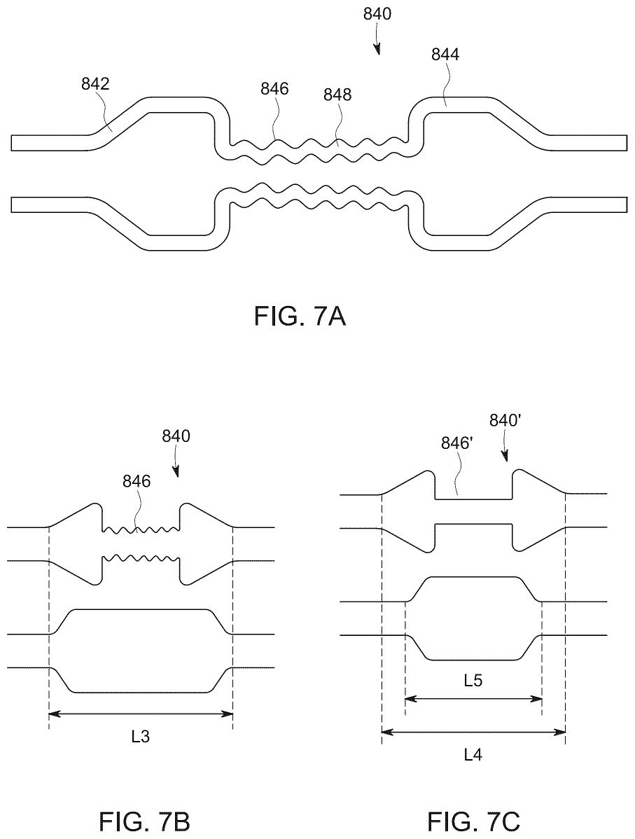

FIG. 7A is a cross-section of a balloon 840 in an uninflated condition. Balloon 840 may be used with a balloon catheter for deploying a prosthetic heart valve, similar to other embodiments of balloon catheters described herein. As with balloons 640 and 740, balloon 840 may include a pre-formed (or “pre-pillowed”) proximal bulge 842 and distal bulge 844, which are separated by an intermediate portion 846 that is configured to receive the prosthetic heart valve thereon. Unlike the other embodiments disclosed herein, the intermediate portion 846 of the balloon 840 includes excess material that forms a plurality of pleats or folds 848. The pleats or folds 848 may provide two separate functions. First, the folds 848 may create greater friction between the balloon 840 and the prosthetic heart valve crimped thereon, particularly compared to an identically formed balloon that has a smooth outer surface without folds. This extra friction may help the prosthetic heart valve mounted on the balloon 840 to more reliably maintain its position after crimping due to the extra friction. Second, as inflation media is passed into the balloon 840 and the balloon 840 begins to expand, the folds 848 may begin to unfold or unfurl. In other words, a length of the intermediate section 846 (e.g. a length that follows the contours of the folds 848) is greater than a length from a distal end of the proximal bulge 842 to a proximal end of the distal bulge 844 when the balloon 840 is uninflated. As shown in FIG. 7B, which compares the uninflated condition of balloon 840 to the inflated condition of the balloon 840, the extra material of the folds 848 allows the length L3 of the balloon 840 to be substantially constant as the balloon 840 expands. In other words, while the natural tendency for the balloon 840 would be to axially foreshorten during expansion, the unfolding of the folds 848 during expansion compensates for this tendency, allowing the balloon 840 to maintain a constant length L3. This should be compared to an alternate version of the balloon 840′, shown in FIG. 7C, that is identical to balloon 840 except that there are no folds on the intermediate portion 846′. Without the folds, the balloon 840′ has a first length L4 in the uninflated condition, but as the balloon 840′ expands, the overall length of the balloon 840′ axially foreshortens to a smaller length L5.

One of the benefits of reducing or eliminating the axial foreshortening of balloon 840 during expansion is that the prosthetic heart valve mounted thereon may experience a smaller degree of foreshortening. In other words, if a prosthetic heart valve is mounted on balloon 840′, as the balloon 840′ expands and foreshortens, it may tend to pull the prosthetic heart valve and cause it to foreshorten along with the balloon. However, the construction of balloon 840 may reduce the amount of foreshortening of the prosthetic heart valve during expansion of the balloon 840, which in turn may help achieve more accurate positioning of the prosthetic heart valve into the native valve annulus.

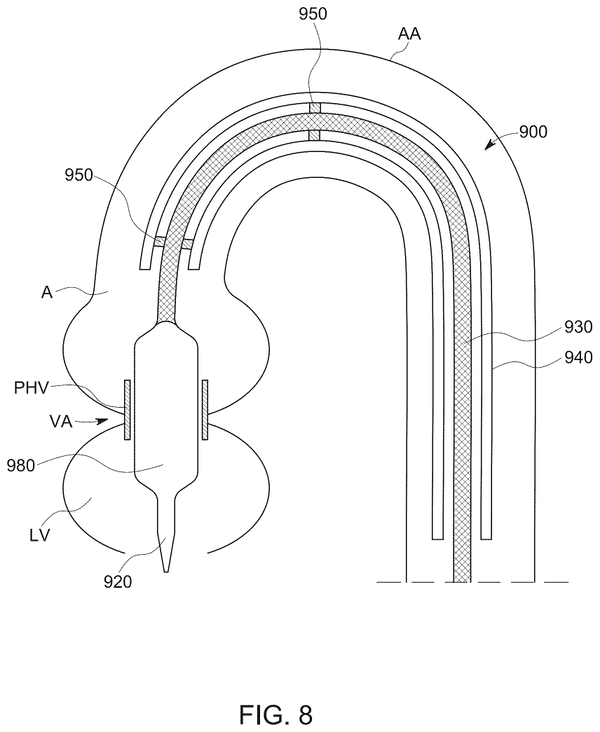

FIG. 8 is a highly schematic cross-section of a delivery device 900 according to another aspect of the disclosure. In FIG. 8, the delivery device 900 has been advanced through the femoral artery, across the aortic arch AA of the aorta A, and a distal end of the delivery device 900 is positioned within the aortic valve annulus VA, with the nosecone 920 of the delivery device 900 extending into the left ventricle LV. As with other delivery devices described herein, delivery device 900 may include a balloon catheter shaft 930 that connects to an expandable balloon 980, for example with an inflation lumen connecting the balloon catheter shaft 930 to the interior of the balloon 980. A balloon-expandable prosthetic heart valve PHV may be crimped over the balloon 980 when it is uninflated, and FIG. 8 illustrates the balloon 980 having been inflated to deploy the prosthetic heart valve PHV into the aortic valve annulus VA.

The delivery device 900 of FIG. 8 may also include an outer catheter shaft 940 through which the balloon catheter shaft 930 extends. In the illustrated embodiment, the outer catheter shaft 940 terminates proximal to the proximal end of the balloon 980. Outer catheter shaft 940 may be steerable, for example via one or more pull wires extending through the wall of the outer catheter shaft 940 to a steering ring mounted to the outer catheter shaft 940. Steerability of the outer catheter shaft 940 may assist with navigation of the delivery device 900, including navigation around the aortic arch AA while minimizing or avoiding contact between the collapsed prosthetic heart valve PHV and the tissue of the aortic arch AA. In the embodiment of FIG. 8, delivery device 900 includes one or more spacers 950 extending between the balloon catheter shaft 930 and the outer catheter shaft 940. Although two spacers 950 are shown, it should be understood that one, two, or more than two spacers 950 may be provided along the length of the delivery device 900. The spacers 950 may be formed from any suitable material that has enough rigidity to maintain the desired clearance between the balloon catheter shaft 930 and the outer shaft 940. Suitable materials may include soft polymers that can be overmolded onto the balloon catheter shaft 930, with the soft nature of the material allowing for desired amounts of flexibility. In some examples, the spacers 950 may be formed as annular disks (or cylindrical disks with a central through-hole). For example, each spacer 950 may include an interior cylindrical or circular through-hole that has a diameter that is about equal to the outer diameter of the balloon catheter shaft 930, and may extend to an outer diameter that is about equal to the inner diameter of the balloon catheter shaft 940. With this configuration, each spacer 950 receives the balloon catheter shaft 930 therethrough, and includes an outer perimeter in contact with the inner diameter of the outer shaft 940. In some embodiments, each spacer 950 is fixed to both the balloon catheter shaft 930 and the outer shaft 940. However, in other embodiments, each spacer 950 is fixed only to the balloon catheter shaft 930 (or only to the outer shaft 940) to allow for axial and/or rotational movement of the balloon catheter shaft 930 relative to the outer shaft 940. With the configuration described in connection with FIG. 8, the spacers 950 may force the balloon catheter shaft 930 and the outer shaft 940 to maintain coaxial positioning relative to each other, with a clearance between the two shafts equal to the size of the spacers 950.

The spacers 950 may provide one or more benefits when using the delivery device 900 to deliver and deploy prosthetic heart valve PHV via expansion of balloon 980. For example, if the outer shaft 940 is steerable, by steering the outer shaft 940 to be generally aligned (e.g. coaxial) with the native aortic valve annulus VA, the spacers 950 may force the balloon catheter shaft 930 to be coaxial with the outer shaft 940, and thus also coaxial with the native aortic valve annulus VA. The spacers 950 may also help resist a change in relative positioning between the balloon catheter shaft 930 and the outer shaft 940. For example, as the balloon 980 expands and deploys the prosthetic heart valve PHV into the native valve annulus VA, forces from the balloon expansion and from contact between the prosthetic heart valve PHV and the native valve annulus VA may tend to change the position of the balloon catheter shaft 930 relative to the outer shaft 940. However, the inclusion of the spacers 950 may resist such relative movement, thus increasing the overall stability of the delivery device 900 and the accuracy of the placement of the prosthetic heart valve PHV within the native valve annulus VA.

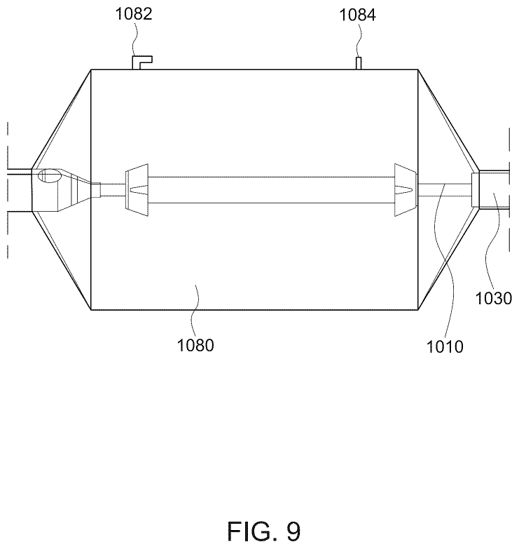

FIG. 9 illustrates the distal end of a delivery device according to another aspect of the disclosure. In particular, FIG. 9 illustrates the distal end of a balloon catheter shaft 1030 coupled to a balloon 1080 (the balloon 1080 being shown in an inflated condition in FIG. 9), with an inner shaft 1010 passing through the balloon 1080 and to an atraumatic distal tip or nosecone (not visible in FIG. 9). Although not shown in FIG. 9, it should be understood that a balloon expandable prosthetic heart valve would be crimped over balloon 1080 when the balloon 1080 is uninflated for delivery, and the prosthetic heart valve would expand with the balloon 1080 as inflation media is passed through the balloon catheter shaft 1030 into the interior of the balloon 1080. The balloon 1080 in FIG. 9 includes one or more features to temporarily couple the balloon 1080 to the prosthetic heart valve crimped thereon.

In the specific embodiment shown in FIG. 9, the balloon 1080 includes a distal or inflow connector 1082 and a proximal or outflow connector 1084. In this embodiment, the distal connector 1082 is fixed to an outer surface of the balloon 1080, near a distal end thereof, and may have a general “L”-shape with one member extending radially outward of the balloon 1080 and a second member extending proximally. The proximal 1084 is also fixed to the balloon 1080, near a proximal end thereof, and may have a single member extending radially outward from the balloon 1080, for example as a simple short rod. The proximal and distal connectors 1082, 1084 may be formed of any suitable material, preferably including materials that have enough rigidity to maintain contact with the prosthetic heart valve as the balloon 1080 expands. In some embodiments, the connectors 1082, 1084 may be formed of an amide-based polymer, for example to allow for chemical bonding to the balloon 1080. In some embodiments, the connectors 1082, 1084 may be formed of a material with a higher melt temperature than that of the balloon 1080. In such embodiments, the connectors 1082, 1084 may be overmolded onto the parison prior to blow molding the balloon 1080. During blow molding, the connectors 1082, 1084 may move outward, but remain geometrically unchanged. In some embodiments, it may be possible to couple the connectors 1082, 1084 to the balloon 1080 after blow molding the balloon 1080. In such embodiments, it may be possible to overmold the connectors 1082, 1084 onto the balloon, although the thinness of the wall of the balloon 1080 may make such overmolding difficult. But, even if more difficult, such a process may lead to better precision in placement of the connectors 1082, 1084 since the blow molding of the balloon 1080 is already completed prior to attachment of the connectors 1082, 1084.

In use, when the prosthetic heart valve is crimped over the uninflated balloon 1080, the two connectors 1082, 1084 may contact struts that form the frame of the prosthetic heart valve (which may be any prosthetic heart valve described herein, or any other balloon-expandable prosthetic heart valve). As the balloon 1080 expands, the contact between the connectors 1082, 1084 and the prosthetic heart valve may help to ensure that the prosthetic heart valve maintains desired positioning relative to the balloon 1080 during expansion. For example, in some embodiments, the bottom inflow apex of a cell of the frame of the prosthetic heart may be hooked over the distal end of distal connector 1082, and a top outflow apex of a cell of the frame may be hooked over the proximal end of the proximal connector 1084. With this configuration, as the balloon 1080 expands, the connectors 1082, 1084 limit the ability of the prosthetic heart valve to foreshorten because the connectors 1082, 1084 prevent sliding axial motion of the ends of the frame relative to the balloon. However, in other embodiments, it may be desirable to allow only one end (e.g. the outflow end) of the prosthetic heart valve to foreshorten, in which case the top outflow apex of a cell of the frame may be in contact (instead of hooked over) the distal end of the proximal connector 1084, with such contact generally helping maintain positional (e.g. rotational) stability of the outflow end of the prosthetic heart valve relative to the balloon 1080, but not limiting foreshortening of the outflow end of the prosthetic heart valve. Other positioning is possible, for example with the “L”-shaped distal connector 1082 hooking over a strut at or near the inflow end of the frame of the prosthetic heart valve, to similarly help stability the inflow end of the prosthetic heart valve without necessarily limiting foreshortening of the inflow end.

Although FIG. 9 illustrates two specific connectors 1082, 1084 on the balloon 1080, it should be understood that additional connectors may be provided, for example multiple pairs connectors 1082 and 1084 may be provided along the circumference of the balloon even though only one pair is shown. In other embodiments, either connector 1082, 1084 may be omitted, or additional connectors may be provided (e.g. between connectors 1082, 1084) to provide additional stability. It should also be understood that the connectors 1082, 1084 may be provided on other embodiments described herein. For example, balloon 1080 may be provided with pre-bulged or pre-pillowed sections like that shown in connection with FIGS. 5-7C, and the connectors may be provided at the ends of the intermediate (non-pillowed) section of the balloon, with the opposite ends of the prosthetic heart valve contacting the shoulders formed by the pre-bulged or pre-pillowed sections when the prosthetic heart valve is crimped over the uninflated balloon 1080.

The delivery devices described above are generally described with a focus on the distal end of the device. However, it should be understood that any of the embodiments described herein may include proximal ends that include a handle for controlling various aspects of the process, including actuators for controlling the steering of an outer shaft of the prosthetic heart valve, actuators that provide for rotation of the balloon catheter shaft in order to align commissures of the prosthetic heart valve with commissures of the native heart valve, one or more ports for introducing fluid (e.g. flush ports and/or inflation ports) into the system, etc. as is known in the art. Further, although one example balloon-expandable prosthetic heart valve is described herein, it should be understood that other balloon expandable prosthetic heart valves may be equally suitable for use with any of the delivery device (or delivery device features) described herein. Still further, although various features are described herein as parts of individual embodiments, it should be understood that features of different embodiments may be combinable with each other. For example, the unevenly sized pre-bulges of FIG. 5 may be applied to any of the other balloons described herein. Similarly, the inflation lumen modifier described in connection with FIG. 6 may be applied to any of the other balloons described herein. The pleats or folds 848 of the intermediate section 846 of the balloon of FIG. 7A may similarly be combined with any other balloon described herein. The spacers 950 described in connection with FIG. 8 may be used with the delivery device of any other embodiment described herein. Similarly, the connectors 1082, 1084 (and variants thereof described above) may be applied to any of the balloons described herein. Finally, it should be understood that more than two (including all) of these features may be combined in a single embodiment, and the benefits of each feature may apply to the combined device. These benefits are generally directed to more accurate deployment of the prosthetic heart valve from the collapsed condition to the expanded condition, including by limiting foreshortening (particularly at the inflow end) of the prosthetic heart valve, or otherwise stabilizing the position of the prosthetic heart valve as it expands during deployment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A delivery device for delivering a prosthetic heart valve, the delivery device comprising:
a balloon catheter shaft having a distal end;
an inner shaft extending through the balloon catheter in a proximal-to-distal direction;
an atraumatic distal tip positioned at a distal end of the inner shaft; and
a balloon positioned between the distal end of the balloon catheter shaft and the atraumatic distal tip, the balloon having an inflated condition and an uninflated condition,
wherein in the uninflated condition of the balloon, the balloon includes a proximal bulge, a distal bulge, and an intermediate section between the proximal bulge and the distal bulge, the intermediate section having a diameter that is smaller than a diameter of the proximal bulge and smaller than a diameter of the distal bulge, the distal bulge extending a first length in the proximal-to-distal direction, and the proximal bulge extending a second length in the proximal-to-distal direction, the first length being different than the second length.

2. The delivery device of claim 1, wherein the balloon catheter shaft includes an inflation lumen therein in fluid communication with an interior volume of the balloon such that pushing inflation media through the inflation lumen in the proximal-to-distal direction results in the inflation media entering the proximal bulge before entering the distal bulge.

3. The delivery device of claim 1, wherein the first length is between about 1.1 and about 1.5 times longer than the second length.

4. The delivery device of claim 3, wherein in the inflated condition of the balloon, the diameters of the proximal bulge, distal bulge, and intermediate section are all substantially equal.

5. The delivery device of claim 1, wherein an inflation lumen modifier is positioned on the inner shaft and occupies a position within an interior volume of the balloon that is axially aligned with a position of the intermediate section.

6. The delivery device of claim 1, wherein in the uninflated condition of the balloon, the intermediate section of the balloon has a plurality of folds or pleats so that a length of the intermediate section is greater than a length from a distal end of the proximal bulge to a proximal end of the distal bulge.

7. A delivery device for delivering a prosthetic heart valve, the delivery device comprising:
a balloon catheter shaft having a distal end;
an inner shaft extending through the balloon catheter in a proximal-to-distal direction;
an atraumatic distal tip positioned at a distal end of the inner shaft; and
a balloon positioned between the distal end of the balloon catheter shaft and the atraumatic distal tip, the balloon having an inflated condition and an uninflated condition,
wherein in the uninflated condition of the balloon, the balloon includes a proximal bulge, a distal bulge, and an intermediate section between the proximal bulge and the distal bulge, the intermediate section having a diameter that is smaller than a diameter of the proximal bulge and smaller than a diameter of the distal bulge,
wherein an inflation lumen modifier is positioned on the inner shaft and occupies a position within an interior volume of the balloon that is axially aligned with a position of the intermediate section.

8. The delivery device of claim 7, wherein the inflation lumen modifier is a solid cylindrical member, the inner shaft passing through an interior of the inflation lumen modifier.

9. The delivery device of claim 7, wherein in the uninflated condition of the balloon, a total fillable interior volume of the intermediate section of the balloon is smaller compared to the total fillable interior volume of the intermediate section of the balloon in an absence of the inflation lumen modifier.

10. The delivery device of claim 7, wherein when the balloon transitions from the uninflated condition to the inflated condition, the proximal bulge, the intermediate section, and the distal bulge expand at a substantially equal rate of expansion.

11. The delivery device of claim 7, wherein in the uninflated condition of the balloon, the intermediate section of the balloon has a plurality of folds or pleats so that a length of the intermediate section is greater than a length from a distal end of the proximal bulge to a proximal end of the distal bulge.

12. A delivery device for delivering a prosthetic heart valve, the delivery device comprising:
a balloon catheter shaft having a distal end;
an inner shaft extending through the balloon catheter in a proximal-to-distal direction;
an atraumatic distal tip positioned at a distal end of the inner shaft; and
a balloon positioned between the distal end of the balloon catheter shaft and the atraumatic distal tip, the balloon having an inflated condition and an uninflated condition,
wherein in the uninflated condition of the balloon, the balloon includes a proximal bulge, a distal bulge, and an intermediate section between the proximal bulge and the distal bulge, the intermediate section having a plurality of folds or pleats so that a length of the intermediate section is greater than a length from a distal end of the proximal bulge to a proximal end of the distal bulge, the intermediate section having a diameter that is smaller than a diameter of the proximal bulge and smaller than a diameter of the distal bulge.

13. The delivery device of claim 12, wherein as the balloon transitions from the uninflated condition to the inflated condition, the plurality of folds or pleats of the intermediate section unfold or unfurl.

14. The delivery device of claim 13, wherein in the uninflated condition of the balloon, the balloon has a first length between a proximal end of the proximal bulge and a distal end of the distal bulge, and in the inflated condition of the balloon, the balloon has a second length between the proximal end of the proximal bulge and the distal end of the distal bulge, the first length being about equal to the second length.

15. The delivery device of claim 12, wherein in the uninflated condition of the balloon, contact between the plurality of folds or pleats with an interior surface of a prosthetic heart valve results in greater friction compared to friction between the interior surface of the prosthetic heart valve with an identically formed balloon that has an intermediate portion with a smooth outer surface without folds.

16. The delivery device of claim 12, wherein the delivery device further includes an outer shaft extending over the balloon catheter shaft in the proximal-to-distal direction, wherein at least one spacer is coupled to an outer surface of the inner shaft and is in contact with an inner surface of the outer shaft so that the inner shaft is coaxial with the outer shaft.

17. The delivery device of claim 16, wherein the at least one spacer includes a plurality of spacers positioned at spaced distances along the inner shaft.

18. The delivery device of claim 16, wherein the at least one spacer is fixed to the inner shaft.

19. The delivery device of claim 16, wherein the spacer is annularly shaped, with an interior circular hole through which the inner shaft passes, and an outer circumference in contact with the inner surface of the outer shaft.

20. The delivery device of claim 16, wherein a distal end of the outer shaft terminates proximal to a proximal end of the balloon.
